(19) United States
(12) Patent Application Publication
(10) Pub. No.: US 2018/0110840 A1
(43) Pub. Date: Apr. 26, 2018

(54) USE OF OVOTRANSFERRIN TO PROMOTE BONE HEALTH

(71) Applicant: THE GOVERNORS OF THE UNIVERSITY OF ALBERTA, Edmonton (CA)

(72) Inventors: Jianping WU, Edmonton (CA); Nan SHANG, Edmonton (CA)

(73) Assignee: THE GOVERNORS OF THE UNIVERSITY OF ALBERTA, Edmonton (CA)

(21) Appl. No.: 15/787,772

(22) Filed: Oct. 19, 2017

Related U.S. Application Data
(60) Provisional application No. 62/410,535, filed on Oct. 20, 2016.

(51) Int. Cl. A61K 38/40; A61K 9/00

(52) U.S. Cl. CPC A61K 38/40; A61K 9/0053

(57) ABSTRACT

Described herein is the use of ovotransferrin to promote bone health. It has been discovered that we found that ovotransferrin can promote cell proliferation and differentiation of osteoblasts. Furthermore, ovotransferrin can inhibit the expression of receptor activator of nuclear factor kappa-B ligand (RANKL) in osteoblasts as well as promote the expression of alkaline phosphatase (ALP), type I collagen, and osteoprotegerin (OPG) in osteoblasts. These unique properties make ovotransferrin a useful treatment option for subjects afflicted with bone disease such as, for example, osteoporosis.

### CROSS REFERENCE TO RELATED APPLICATIONS

This application claims priority upon U.S. provisional application Ser. No. 62/410,535, filed Oct. 20, 2016. This application is hereby incorporated by reference in its entirety for all of its teachings.

### BACKGROUND

Osteoporosis is defined as a skeletal disorder described by an impairment of bone mass, strength, and microarchitecture, which causes severe and localized pain, increased bone fractures, and reduced height. Osteoporosis is caused by an imbalance between bone formation and bone resorption, which is regulated by the activities of osteoblasts and osteoclasts, respectively. Osteoporosis is a serious public health concern, affecting more than 200 million people worldwide (International Osteoporosis Foundation, 2016) and causing more than 1.5 million fractures annually (Masi, 2008). Moreover, osteoporosis is also a major cause of morbidity and health expenditure in aging populations (Cummings, and Melton, 2002; Naot et al., 2005). Currently, antiresorptive medications such as bisphosphonates, selective estrogen receptor modulators and hormones are the main pharmacological treatment options for osteoporosis patients (Chen and Sambrook, 2012); however, these agents are limited in their ability to restore bone mass and are associated with side effects.

### SUMMARY

Described herein is the use of ovotransferrin to promote bone health. It has been discovered that ovotransferrin can promote cell proliferation and differentiation of osteoblasts. Furthermore, ovotransferrin can inhibit the expression of receptor activator of nuclear factor kappa-B ligand (RANKL) in osteoblasts as well as promote the expression of alkaline phosphatase (ALP), type I collagen, and osteoprotegerin (OPG) in osteoblasts. These unique properties make ovotransferrin a useful treatment option for subjects afflicted with bone disease such as, for example, osteoporosis.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the alamar blue assay for MC3T3-E1 cells treated with ovotransferrin (10 μg/mL to 1000 μg/mL) and lactoferrin (1000 μg/mL).

FIG. 2 shows the BrDU incorporation assay for MC3T3-E1 cells treated with ovotransferrin (10 μg/mL to 1000 μg/mL) and lactoferrin (1000 μg/mL).

FIG. 3 shows the expression level and western blot band of ALP for MC3T3-E1 cells treated with ovotransferrin (1 μg/mL to 1000 μg/mL) and lactoferrin (1000 μg/mL).

FIG. 4 shows the expression level and fluoresce microscopy images of collagen I for MC3T3-E1 cells treated with ovotransferrin (10 μg/mL to 1000 μg/mL) and lactoferrin (1000 μg/mL).

FIG. 5 shows the expression level and western blot band of RANKL for MC3T3-E1 cells treated with ovotransferrin (1 μg/mL to 1000 μg/mL) and lactoferrin (1000 μg/mL).

FIG. 6 shows the expression level and western blot band of OPG for MC3T3-E1 cells treated with ovotransferrin (1 μg/mL to 1000 μg/mL) and lactoferrin (1000 μg/mL).

FIG. 7 shows the calcium deposits (mineralization) level of MC3T3-E1 cells treated with ovotransferrin (1 μg/mL to 1000 μg/mL) and lactoferrin (1000 μg/mL).

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise.

“Optional” or “optionally” means that the subsequently described event or circumstance can or cannot occur, and that the description includes instances where the event or circumstance occurs and instances where it does not.

“Subject” refers to mammals including, but not limited to, humans, non-human primates, sheep, dogs, rodents (e.g., mouse, rat, etc.), guinea pigs, cats, rabbits, cows, horses, and non-mammals including chickens, amphibians, and reptiles, who are at risk for or have been diagnosed with a condition that causes bone loss and benefits from the methods and compositions described herein.

As used herein, the term “about” is used to provide flexibility to a numerical range endpoint by providing that a given value may be “a little above” or “a little below” the endpoint without affecting the desired result.

The term “treat” as used herein is defined as maintaining or reducing the symptoms of a pre-existing condition when administered ovotransferrin when compared to the same symptom in the subject prior to the administration of ovotransferrin to the subject.

The term “prevent” is defined as eliminating the onset of a bone disease (e.g., bone loss) in a subject, where the bone disease did exist in the subject.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Application of Ovotransferrin for Bone Health

As a dynamic tissue, bone is subjected to a continuous process of bone remodeling (Zaidi, 2007; Cohen, 2006). Bone remodeling is a highly coordinated process responsible for bone resorption and formation throughout life (Raggatt and Partridge, 2010). The process of remodeling is not only to adapt bone strength and repair damaged bone, but also to maintain mineral homeostasis (Redlich and Smolen, 2012). Thus, a proper balance between bone formation and resorption has a critical influence on ultimate bone health. Three main types of bone cells contribute to the remodeling process: osteoblasts, osteocytes, and osteoclasts (Silverman and Abrahamsen, 2016). Osteoblasts are responsible for bone formation through cell differentiation and mineralization. After deposit on the bone matrix, osteoblasts will be encased by the matrix and become mature osteocytes or remain on the surface of the bone as bone-lining cells (Silverman and Abrahamsen, 2016; Datta et al., 2007). In contrast, osteoclasts have the function to disrupt the old bone tissue (Crockett et al., 2011). Hence, the most common strategy for osteoporosis prevention and treatment of bone disease such as osteoporosis is to stimulate the activity of osteoblasts while inhibiting the activity of osteoclast.

It has been discovered herein that ovotransferrin can promote bone formation and be useful in enhancing bone health in patients. Ovotransferrin is an iron-binding glycoprotein found in avian egg white and in avian serum, belonging to the family of transferrin iron-binding glycoproteins (Giansanti et al., 2012; Mine, 2008). Ovotransferrin is a disulfide-rich single chain glycol-protein containing 686 residues with a molecular mass of about 78-80-kDa, and a glycan chain attached to the C-terminal domain. Ovotransferrin belongs to the transferrin family, which are two-lobe proteins (bilabial molecule) with a strong site for iron binding located in each lobe. It contains 15 disulfide bridges, and there are 6 homologous bridges in each half of the molecule and 3 extra bridges which occur only in the C-terminal half. These disulfide bridges are believed to play a role in ovotransferrin's resistance to enzymatic digestion.

In one aspect, ovotransferrin can be administered to a subject to treat or prevent a condition that causes loss of bone mass, or to initiate the de novo turnover of bone. The subject can either be experiencing bone loss or be at risk for such a condition, or conversely may be unable to initiate bone remodeling. To determine whether a subject is experiencing metabolic bone disease, numerous tests, such as bone density testing, a battery of genetic tests, a subject's medical history, and the subject's family medical history, can be used to make this determination.

In one aspect, the subject has a condition that promotes bone loss. Examples of such conditions include, but are not limited to, osteoporosis, Paget's disease, osteolytic tumors, Rheumatoid Arthritis, Psoriatic Arthritis, Ankylosing Spondylitis, Osteoarthritis, osteoporosis, osteosclerotic tumors, hypercalcemia, osteopenia including drug induced osteopenia, or a combination thereof.

In certain aspects, bone loss can be reduced by contacting the bone with the compositions described herein. In other aspects, the compositions described herein can be administered to a subject to prevent bone fractures and to strengthen bones. In other aspects, the compositions described herein can be administered to a subject to stimulate de novo bone turnover and to improve bone balance.

In each of these aspects, administration may be via oral administration, injection including intramuscular or subcutaneous injection, transdermal or transcutaneous routes or via nasal administration. In the case of oral administration, digestion products of ovotransferrin can also be useful in the methods described herein for promoting bone health.

Ovotransferrin can be formulated in any excipient the biological system or entity can tolerate to produce pharmaceutical compositions. Examples of such excipients include, but are not limited to, water, aqueous hyaluronic acid, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. For example, the pH of the composition is from about 5 to about 8, which is suitable for topical applications. Additionally, the pharmaceutical compositions can include carriers, thickeners, diluents, preservatives, surface active agents and the like in addition to the compounds described herein.

In other aspects, ovotransferrin can be incorporated into foodstuffs, medication, or additional potable, ingestible, or edible compositions.

It will be appreciated that the actual preferred amounts of ovotransferrin in a specified case will vary according to the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Bone remodeling is regulated through RANK/RANKL/OPG pathway (Kearns, Khosla and Kostenuik, 2008). Osteoblasts are the primary source of receptor activator of nuclear factor κB ligand (RANKL), which binds to RANK on osteoprogenitor cells, stimulating the differentiation of osteoclasts and activating bone resorption (Rucci, 2008). The activity of RANKL is antagonized by osteoprotegerin (OPG), which competitively binds to RANK, and therefore the balance of OPG and RANKL will determine the extent of bone resorption (Silverman and Abrahamsen, 2016).

It has been discovered that ovotransferrin can promote cell proliferation and differentiation of osteoblasts. Furthermore, ovotransferrin can inhibit the expression of receptor activator of nuclear factor kappa-B ligand (RANKL) in osteoblasts as well as promote the expression of alkaline phosphatase (ALP), type I collagen, and osteoprotegerin (OPG) in osteoblasts. Moreover, ovotransferrin can promote cell proliferation and differentiation of osteoblasts. All results are presented below in the Examples.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Ovotransferrin (Conalbumin from chicken egg white) with 98% purity was purchased from Sigma Chemicals Co (St Louis, Mo., USA). Dulbecco's modified Eagle medium (DMEM) and Fetal Bovine Serum (FBS) were obtained from Gibco/Invitrogen (Carlsbad, Calif., USA). AlamarBlue Cell Viability Reagent and BrdU Labeling Reagent were purchased from ThermoFisher Scientific Inc. (Waltham, Mass., USA). Collagen I alpha Antibody was purchased from Novus Biological Canada ULC (Oakville, ON, Canada). Hoechst 33342 (Trihydrochloride), Rabbit anti-Mouse IgG (H+L) Secondary Antibody, and Goat anti-Rabbit IgG (H+L) Secondary Antibody were purchased from Molecular Probes (Waltham, Mass., USA). BrdU Mouse antibody was purchased from Cell Signaling Technology Inc. (Danvers, Mass., USA). Triton-X-100 was purchased from VWR International (West Chester, Pa., USA). Goat anti-rabbit and Donkey anti-mouse fluorochrome-conjugated secondary antibodies were purchased from Licor Biosciences (Lincoln, Nebr., USA).
Cell Culture: The murine osteoblastic cell line MC3T3-E1 (subclone 4, ATCC CRL-2593) was purchased from ATCC (Manassas, Va., USA). Cells were cultured in DMEM supplemented with 10% FBS and penicillin-streptomycin in an incubator under 95% air and 5% CO₂. Cells were subcultured using 0.25% trypsin every 2 to 3 days. All experiments were performed on 80-90% confluent cells grown in tissue culture grade plastic 96 or 48 well plates. To examine the activity of egg white ovotransferrin, the cells were incubated with different concentration of ovotransferrin or lactoferrin (the positive control) at various periods prior to BrDU incorporation, western blotting, and immunofluorescence.
AlamarBlue assay: The cells were seeded in a 96 well tissue culture plates at a concentration of 1×10⁴ cell/well, and incubated in DMEM with 10% FBS until 80%-90% confluence. Cells were treated with different doses of ovotransferrin (10 μg/mL to 1000 μg/mL) and lactoferrin (positive control, at a concentration of 1000 mg/mL). After 24 hours of incubation, the medium was removed, and cells were washed with PBS, 200 μL, medium (DMEM with 10% FBS and penicillin-streptomycin) and 20 μL AlamarBlue reagent were added. After another 4 h of incubation with protection from direct light by covering with aluminum foil, 150 μL cell supernatants were transferred to opaque 96 well plate and fluorescence was read with excitation wavelength of 560 nm and emission wavelength of 590 nm. Each treatment was replicated four times.
BrDU incorporation assay: The cells were seeded on 48 well tissue culture plates at a concentration of 1×10⁴ cell/well, and incubated in DMEM with 10% FBS. After 4 h of incubation, the medium was changed and different concentrations of ovotransferrin or 1000 μg/mL lactoferrin were added. Lactoferrin was also used as a positive control. Following 24 h of incubation, the cells were washed and placed in fresh DMEM with 1% FBS, containing 1% BrDU for 1 h. The cells were then fixed in 70% ethanol for 20 min, treated with 1N hydrochloric acid (HCl) for 20 minutes to antigen exposure, then permeabilized with 0.1% Triton-X-100 in phosphate buffered saline for 5 min and blocked in 1% bovine serum albumin (BSA) in phosphate buffered saline for 60 min, and finally incubated with mouse monoclonal antibody against BrDU (1:1000) at 4° C. All the steps except the addition of primary antibody were performed at room temperature. Following overnight incubation with the primary antibody, the cells were treated with anti-mouse secondary antibody for 30 min in the dark. Nuclei were stained with the Hoechst33342 nuclear dye. Cells were visualized under an Olympus IX81 fluorescent microscope. For each data point, three random fields were chosen. The percentage of nuclei positive for BrDU staining was noted in each field and the mean was calculated.
Western blotting: The cells were seeded on 48 well tissue culture plates at a concentration of 1×10⁴ cell/well, and incubated in DMEM with 10% FBS. The cells were treated with different concentrations of ovotransferrin or 1000 μg/mL lactoferrin for 72 h. After incubation, the culture medium was removed and the cells lysed in boiling hot Laemmle's buffer containing 50 μM dithiothreitol (DTT) and 0.2% Triton-X-100 to prepare samples for western blot as described before (Chakrabarti & Davidge, 2016). These cell lysates were run in SDS-PAGE, blotted to nitrocellulose membranes and immunoblotted with antibodies against alkaline phosphatase (ALP), RANKL and the loading control α-tubulin. Anti-tubulin was used at 0.4 μg/mL, while the others were used at 1 μg/mL. The protein bands were detected by a Licor Odyssey Biolmager and quantified by densitometry using corresponding software (Licor Biosciences). Each band of ALP or RANKL was normalized to its corresponding band of loading control. Cell lysates from untreated cells were loaded onto every gel. The results were expressed as percentage of the corresponding untreated control results.
Immunofluorescence: The immunofluorescence studies were performed similar to previous studies (Majumder et al., 2013). Briefly, cells were fixed in 4% formalin, permeabilized with 0.1% Triton-X-100 in PBS and immunostained through overnight incubation with a rabbit polyclonal antibody against type I collagen. Cells were treated with Alexa Fluor546 (red) conjugated goat anti-rabbit secondary antibody for 30 min in the dark. Nuclei were stained with the Hoechst33342 nuclear dye (1:10000) for 10 min. After washing to remove unbound antibody/dye, the immunostained cells were observed under an Olympus IX81 fluorescence microscope (Olympus, Tokyo, Japan). Images were obtained using Metamorph imaging software (Molecular Devices, Sunnyvale, Calif., USA). Mean fluorescence intensity was calculated from the intensity of the red fluorescent signal (determined by Adobe Photoshop Elements 2.0 software; Adobe Systems Inc., San Jose, Calif., USA) from 3 randomly selected fields per group.
Statistics: All data are presented as mean±SEM (standard error of mean) of between 4 and 7 independent experiments. Data are expressed as percentage of negative control (untreated cells). Data were analyzed using one way analysis of variance (ANOVA) with Dunnett's post-hoc test for comparisons to control. The PRISM 6 statistical software (GraphPad Software, San Diego, Calif.) was used for the analyses. P<0.05 was considered significant.

### Results

### Ovotransferrin Shows No Cytotoxicity on Osteoblasts

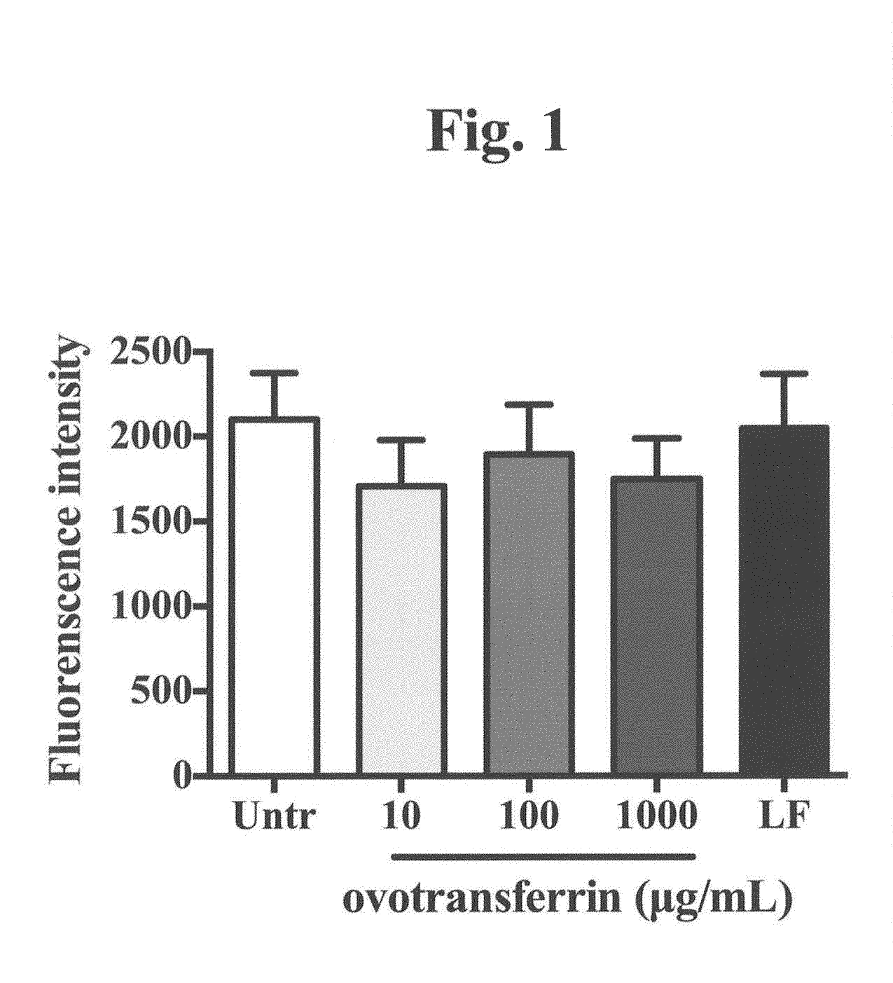

To study the osteoinductive function of ovotransferrin, the cytotoxic effect of ovotransferrin in MC3T3-E1 cell was examined. As shown in FIG. 1, ovotransferrin treatment for 72 h with concentrations ranging from 1 μg/mL to 1000 μg/mL did not cause any significant increase of cell death. These results indicate that ovotransferrin are not cytotoxic to MC3T3-E1 and can be used as a potential function ingredient in following cell study.

### Ovotransferrin Promotes Osteoblasts Proliferation

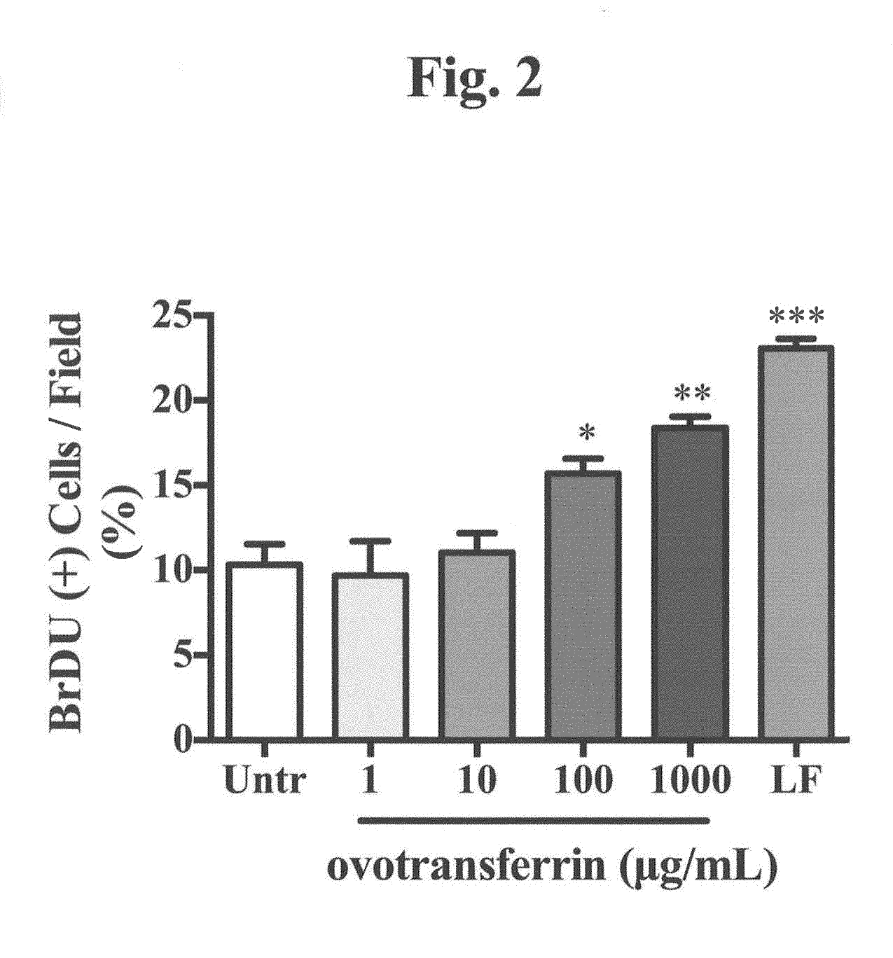

The effect of ovotransferrin on proliferation of osteoblastic cells was investigated. The proliferation effect was determined by BrDU incorporation assay; as shown in FIG. 2, cells treated with concentrations of 100 μg/mL and 1000 μg/mL of ovotransferrin showed significantly increased cell proliferation. The positive control, lactoferrin showed the highest effect on cell proliferation at a concentration of 1000 μg/mL.

### Ovotransferrin Promotes the ALP Expression of Osteoblasts

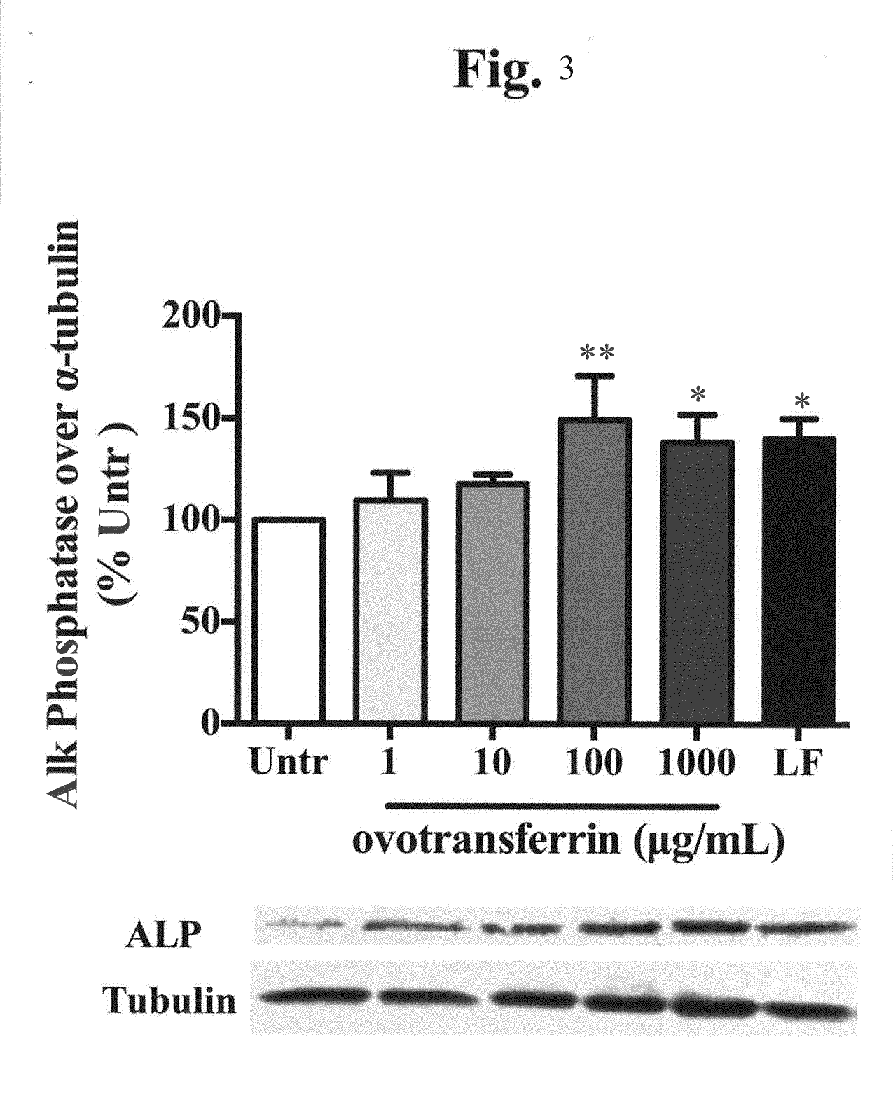

The expression of alkaline phosphatase (ALP) was determined by Western blotting. As shown in FIG. 3, ovotransferrin treatment for 72 h significantly increased ALP expression at concentrations of 100 μg/mL and 1000 μg/mL. Interestingly, the maximal stimulation was observed at 100 μg/mL, but not the highest concentration (1000 μg/mL). As the most important cellular marker that indicates osteoblast differentiation, the increased expression of ALP demonstrated that ovotransferrin may also a potent factor to induce osteoblast differentiation.

### Ovotransferrin Increase Type I Collagen in Osteoblasts

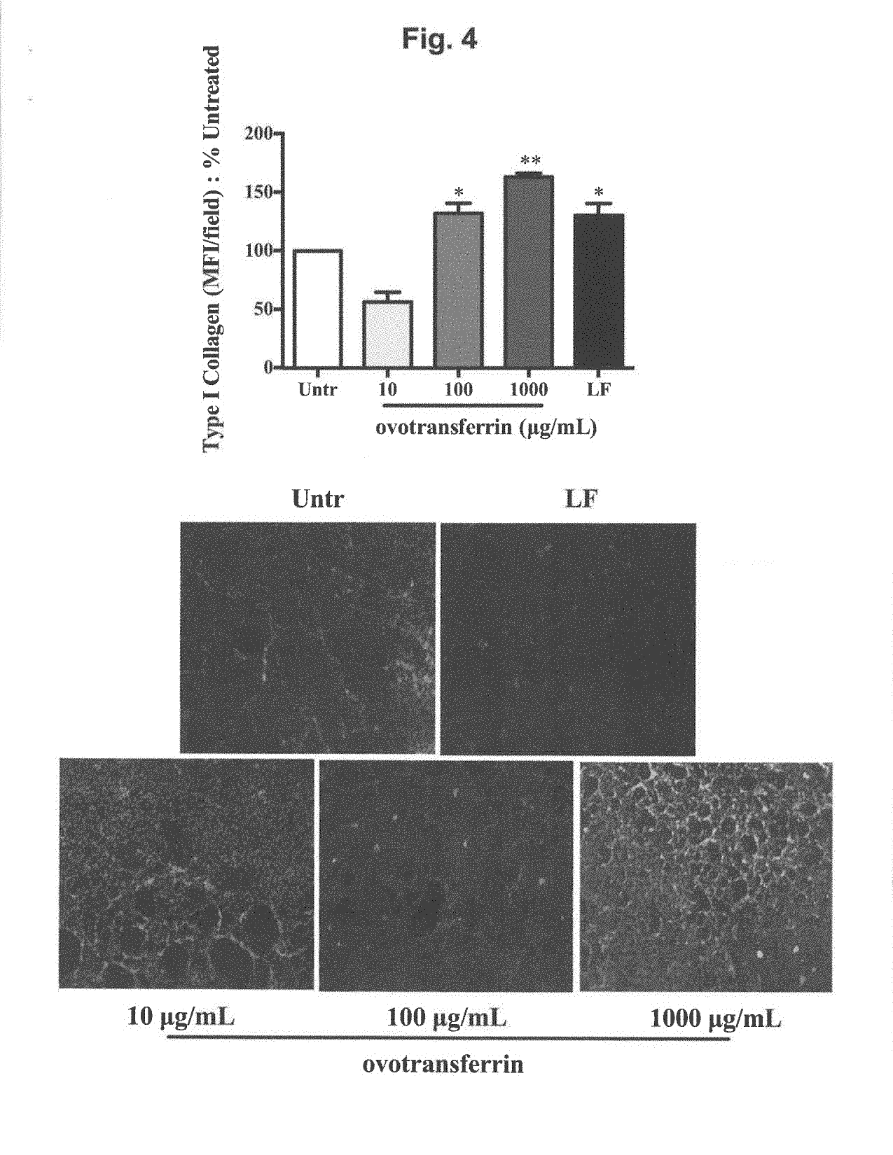

Since type I collagen plays an important role as the structural component in bone formation, expression level of type I collagen was also examined by immunofluorescence. As shown in FIG. 4, expression of type I collagen was significantly increased at ovotransferrin concentrations of 100 μg/mL and 1000 μg/mL, further proved the effects of ovotransferrin in promoting cell differentiation.

### Ovotransferrin Inhibits RANKL Expression

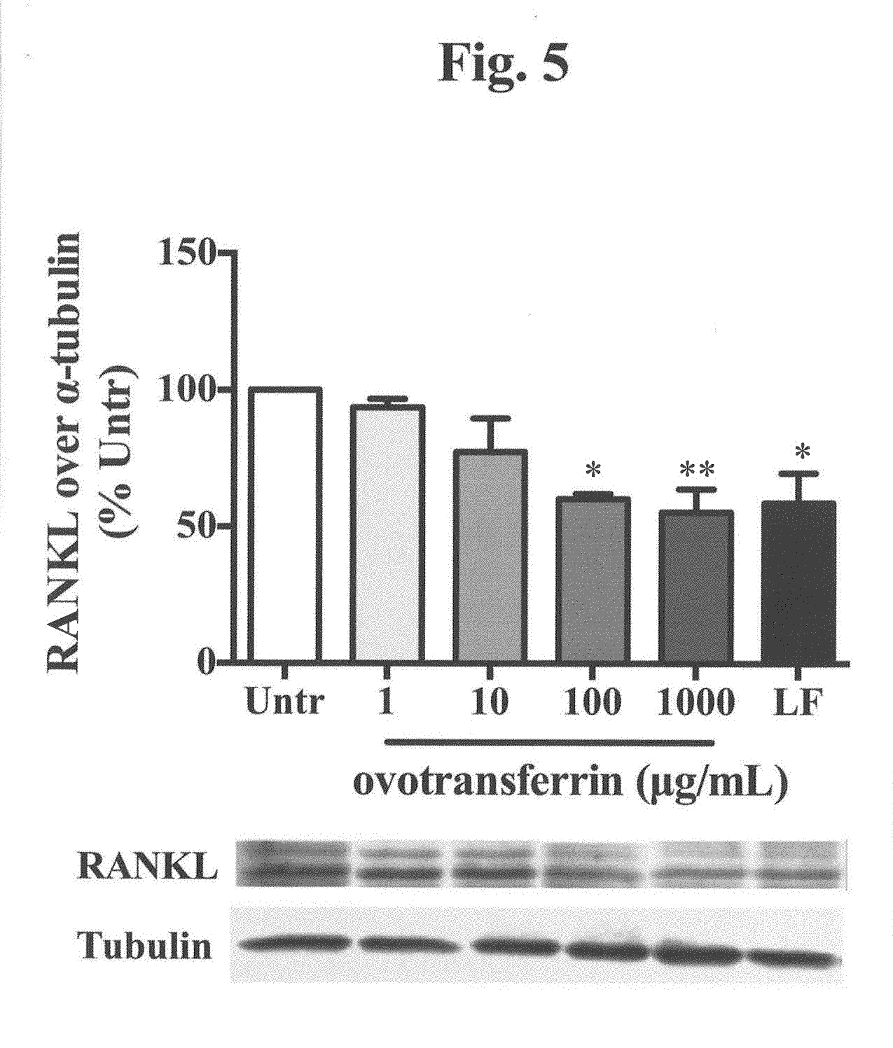

The osteoclastogenesis can only be initiated when RANKL binds to its specific receptor RANK, so the expression level of RANKL is critical in bone remodeling (Rucci, 2008). Since osteoblasts are the primary source of RANKL, the effect of ovotransferrin on RANKL expression of MC3T3-E1 was tested. As shown in FIG. 5, cells treated with 100 μg/mL and 1000 μg/mL significantly inhibited the expression of RANKL similar to lactoferrin, which has been proved to prevent the bone resorption by inhibit the expression of RANKL both in vitro and in vivo (Inubushi et al., 2014; Hou, Xue, and Lin, 2012).

### Ovotransferrin Promote OPG Expression of Osteoblasts

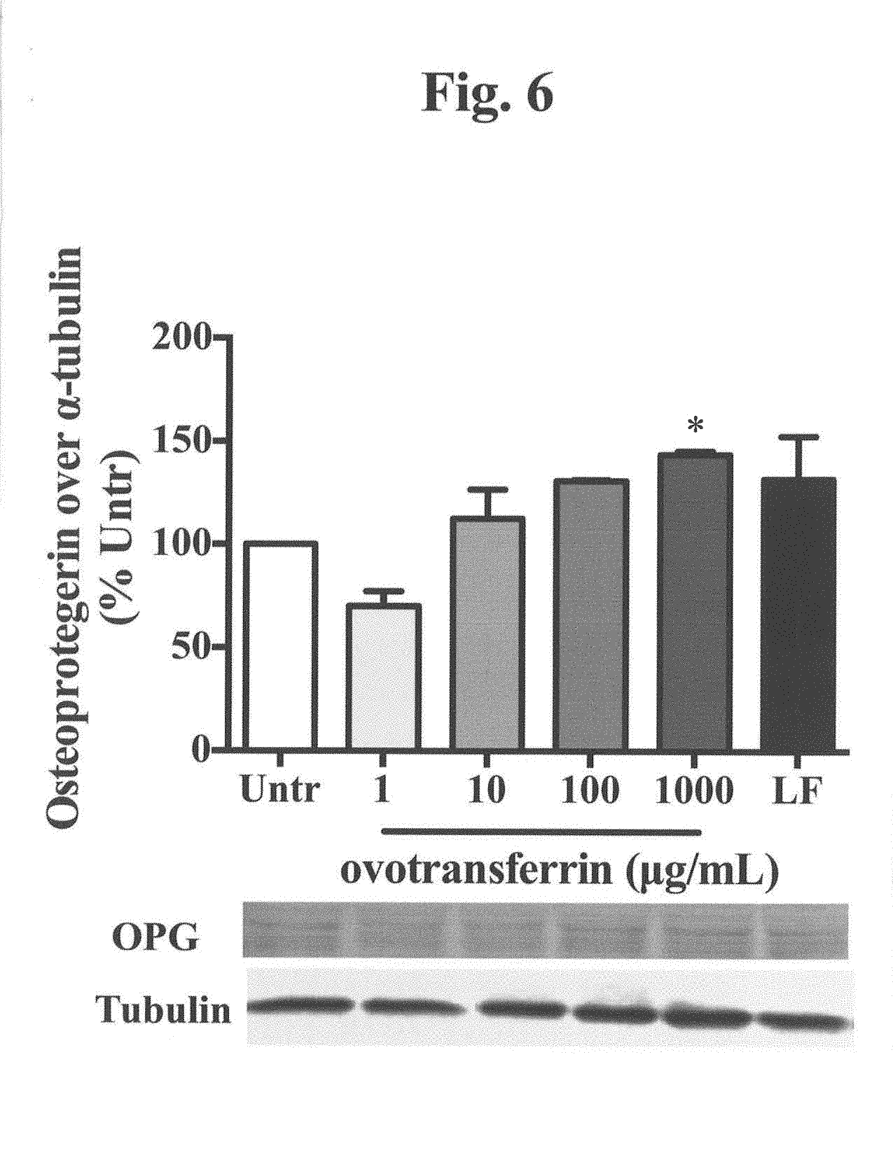

Bone remodeling is regulated through the RANK/RANKL/OPG pathway. OPG, expressed by osteoblast as well, can competitively bind to the receptor RANK to inhibit the activation of osteoclast (Kearns, Khosla and Kostenuik, 2008). The expression level of OPG was also determined after ovotransferrin treatment. As shown in FIG. 6, a significantly increased expression of OPG was only observed at a concentration of 1000 μg/mL compared with the positive control (lactoferrin).

### Mineralization Studies

Mineralization: The mineralization studies were performed similar to previous studies (Reinholz et al., 2000). The degree of mineralization was determined in the 12-well plates using Alizarin Red staining. Cells were incubated with different concentration of ovotransferrin for 10 days. The medium was removed and cells were rinsed twice with PBS. The cells were fixed with ice-cold 70% (v/v) ethanol for 1 h. The ethanol was removed and cells were washed twice with Milli-Q water. The cells were then stained with 1% (w/v) Alizarin Red S in Milli-Q (pH 4.2) for 10 min at room temperature. After washing with Milli-Q water, the samples were observed under light and pictures were photographed.

### Ovotransferrin Promotes Osteoblasts Mineralization

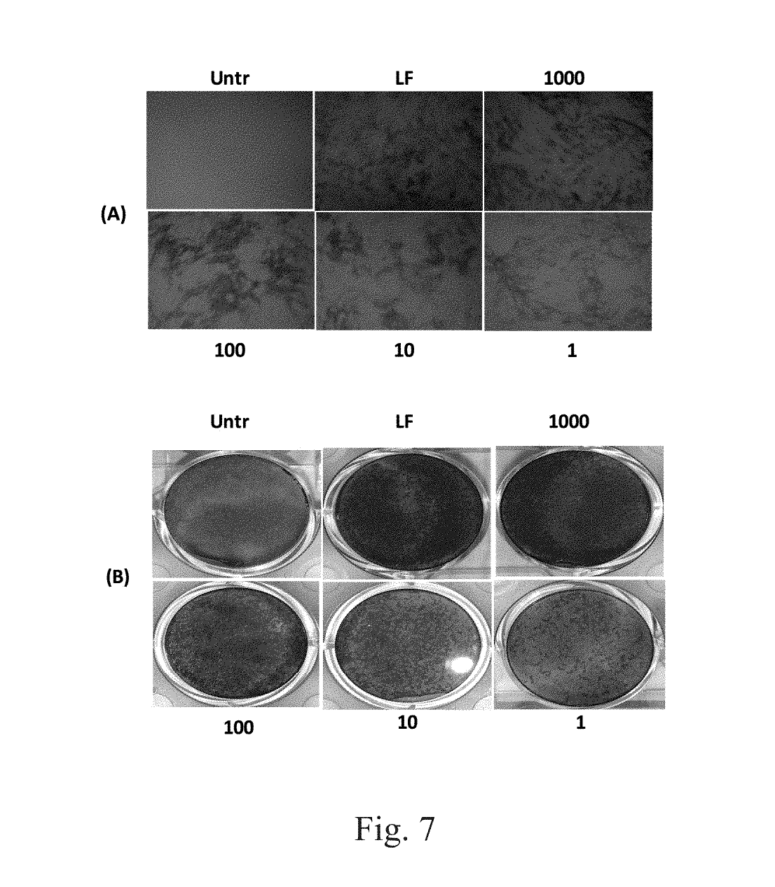

Mineralization is the most important step for bone formation. To examine the effects of ovotransferrin on mineralization, MC3T3-E1 cells were stained for calcium incorporation using Alizarin Red. As shown in FIG. 7, cells treated with different concentration of ovotransferrin (1 μg/mL to 1000 μg/mL) for 10 days increased the amount of the calcium nodules and deposits, which further suggested that ovotransferrin could promote bone formation.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### REFERENCES

Chakrabarti, S., & Davidge, S. T. (2016). Analysis of G-Protein Coupled Receptor 30 (GPR30) on Endothelial Inflammation. Methods Mol Biol, 1366, 503-516.

Crockett J. C., Rogers M. J., Coxon F. P., Hocking L. J., Helfrich M. H. (2011). Bone remodeling at a glance. Journal of Cell Science, 124: 991-998.

Cummings S. R., Melton L. J. (2002). Epidemiology and outcomes of osteoporotic fractures. Lancet, 359:1761-1767.

Datta H. K., Ng W. F., Walker J. A., Tuck S. P., Varanasi S. S. (2007). The cell biology of bone metabolism. Journal of Clinical Pathology, 61: 577-587.

Giansanti F., Leboffe L., Pitari G., Ippoliti R., Antonini G. (2012). Physiological roles of ovotransferrin. Biochinica et Biophysica Acta, 1820: 218-225.

Hou J., Xue Y., Lin Q. (2012). Bovine lactoferrin improves bone mass and microstructure in ovariectomized rats via OPG/RANKL/RANK pathway. Acta Pharmacologica Sinica, 33: 1277-1284.

International Osteoporosis Foundation. (2016). Osteoporosis and Musculoskeletal Disorders. URL http://www.iofbonehealth.org/epidemiology. Accessed Mar. 10, 2016.

Inubushi T., Kawazoe A., Miyauchi M., Kudo Y., Ao M., Ishikado A., Makino T., Takata T. (2011). Molecular mechanisms of the inhibitory effects of bovine lactoferrin on lipopolysaccharide-mediated osteoclastogenesis. Journal of Biological chemistry, 287(28): 23527-23536.

Majumder, K., Chakrabarti, S., Morton, J. S., Panahi, S., Kaufman, S., Davidge, S. T., & Wu, J. P. (2015). Egg-derived ACE-inhibitory peptides IQW and LKP reduce blood pressure in spontaneously hypertensive rats. Journal of Functional Foods, 13, 50-60. doi: 10.1016/j.jff.2014.12.028

Naot D., Grey A., Reid L. R., Cornish J. (2005). Lactoferrin—A novel bone growth factor. Clinical Medicine and Research, 3(2): 93-101.

Raggatt L. J., Partridge N. C. (2010). Cellular and molecular mechanisms of bone remodeling. Journal of Biological Chemistry, 285(33): 25103-25108.

Redlich K., Smolen J. S. (2012). Inflammatory bone loss: pathogenesis and therapeutic intervention. Nature Reviews, 11: 234-250.

Rucci N. (2008). Molecular biology of bone remodeling. Clinical Cases in Mineral and Bone Metabolism, 5(1): 49-56.

Zaidi M. (2007). Skeletal remodeling in health and disease. Nature Medicine, 13(7): 791-801.

What is claimed:

1. A method of treating or preventing loss of bone mass in a subject comprising administering ovotransferrin to the subject having a condition that causes loss of bone mass.

2. The method of claim 1, wherein the condition comprises osteoporosis, Paget's disease, osteolytic tumors, Rheumatoid Arthritis, Psoriatic Arthritis, Ankylosing Spondylitis, Osteoarthritis, hypercalcemia, osteopenia, or any combination thereof.

3. The method of claim 1, wherein the ovotransferrin is administered to the subject by injection or oral administration.

4. The method of claim 1, wherein the ovotransferrin is administered to the subject in a foodstuff.

5. The method of claim 1, wherein the method enhances the growth of osteoblasts.

6. The method of claim 1, wherein the method enhances the expression of type I collagen in osteoblasts.

7. The method of claim 1, wherein the method enhances mineralization in osteoblasts.

8. The method of claim 1, wherein the method enhances the expression of alkaline phosphatase in osteoblasts.

9. The method of claim 1, wherein the method inhibits the expression of receptor activator of nuclear factor kappa-B ligand (RANKL) in osteoblasts.

10. The method of claim 1, wherein the method enhances the expression of osteoprotegrin (OPG) in osteoblasts.

11. A method for promoting bone growth in a subject comprising administering to the subject ovotransferrin.

12. A method for preventing bone fractures in a subject comprising contacting the bone with ovotransferrin.
